(19) United States
(12) Patent Application Publication
(10) Pub. No.: US 2016/0128990 A1
(43) Pub. Date: May 12, 2016

(54) METHODS OF USING RIFAXIMIN IN POSITION EMISSION TOMOGRAPHY (PET) SCANS

(71) Applicant: Salix Pharmaceuticals, Ltd, Raleigh, NC (US)

(72) Inventor: Alan C. Moss, Raleigh, NC (US)

(21) Appl. No.: 14/933,253

(22) Filed: Nov. 5, 2015

Related U.S. Application Data
(63) Continuation of application No. PCT/US2014/038347, filed on May 16, 2014.
(60) Provisional application No. 61/824,780, filed on May 17, 2013.

(51) Int. Cl. A61K 31/437

(52) U.S. Cl. CPC A61K 31/437

(57) ABSTRACT

The present invention provides new methods and kits for the reduction, prevention or inhibition of substrate uptake by non-metastatic, metabolically active cells in a subject who will undergo a position emission tomography (PET) scan.

### RELATED APPLICATIONS

This application is a continuation of International Application No. PCT/US2014/038347 filed May 16, 2014 which claims the benefit of U.S. Provisional Application No. 61/824,780, filed May 17, 2013. The entire contents of each of which are incorporated herein by reference.

### BACKGROUND

Position emission tomography (PET) is a nuclear medicine imaging technique used for the detection of cancer in patients. In the United States, more than two million PET-CT scans were performed in 2010. The basis for the test is the visualization of metabolically-active cells (such as cancer cells) due to their uptake of an administered radionuclide tracer. FDG (2-deoxy-2-[¹⁸F]fluoro-D-glucose) is an exemplary tracer that is administered intravenously (IV) and is widely used in oncology. It is a glucose analog that is taken up by glucose-using cells and becomes trapped there after phosphorylation. Rapidly-proliferating cells, such as cancer cells, take up FDG avidly, allowing the identification of nests of such malignant cells.

Many other metabolically-active cell types also take up FDG, including bacteria, brain, kidneys and immune cells. Thus, a major limitation of current PET-CT studies obtained with FDG is the differentiation between malignant cells and the normal physiologic uptake seen in the brain, heart, intestinal and urinary tracts. Such “false positive” results limit the interpretation of FDG-avid lesions in those areas when malignant cells are being screened for. In particular, the intestinal tract can exhibit intense pockets of FDG uptake. This variable normal intestinal uptake can create difficulties in distinguishing physiological FDG uptake from abnormal uptake in cancer cells.

Accordingly, methods of improving the specificity of substrate uptake and/or methods of reducing background image noise are needed for carrying out more accurate diagnoses based on PET-CT scan images, particularly those of the intestinal tract.

### SUMMARY

Embodiments are directed to a method of reducing the uptake of a substrate by non-metastatic cells in the intestinal tract of a subject, wherein the method includes administering a composition comprising rifaximin to the subject prior to administration of the substrate. In some embodiments, the substrate uptake is reduced by about 10% to 50% relative to a subject that is not administered rifaximin. In some embodiments, the substrate uptake is reduced by about 20% to 35% relative to a subject that is not administered rifaximin. In some embodiments, the subject will undergo a positron emission tomography (PET) scan.

Embodiments are also directed to a method of reducing the risk of false positive diagnosis in a subject undergoing a positron emission tomography (PET) scan, wherein the method includes administering to the subject a composition comprising rifaximin prior to administration of the PET scan. In some embodiments, administration of the composition results in a reduction in the uptake of a substrate by non-metastatic cells in the intestinal tract of the subject. In some embodiments, the substrate uptake is reduced by about 10% to 50% relative to a subject that is not administered rifaximin. In some embodiments, the substrate uptake is reduced by about 20% to 35% relative to a subject that is not administered rifaximin.

In any of the foregoing embodiments, administration of the composition can result in a reduction in the risk of a false positive diagnosis relative to that of a subject that is not administered rifaximin prior to a PET scan.

In some embodiments, the substrate is a radiolabeled sugar analog. For example, the substrate can be radiolabeled with ¹¹C, ¹³N, ¹⁵O, ¹⁸F, ⁸²Rb, or combinations thereof.

In some embodiments, the substrate is 2-deoxy-2-[¹⁸F]fluoro-D-glucose (FDG).

In any of the foregoing embodiments, the subject can be administered rifaximin at a dose of about 10 mg to about 6000 mg per day.

In some embodiments, the subject is administered rifaximin at a dose of from about 10 mg to about 6000 mg; from about 50 mg to about 2500 mg BID; from about 50 mg to about 2000 mg TID; 200 mg TID; 200 mg BID or 200 mg QD.

In some embodiments, the subject is administered rifaximin at a dose of about 550 mg, 600 mg or 1650 mg TID, QD or BID.

In some embodiments, the subject is administered rifaximin at a dose of about 550 mg BID.

In some embodiments, the subject is administered rifaximin at a dose of about 1100 mg per day.

In some embodiments, the subject is administered rifaximin as a solid dispersion, wherein the solid dispersion comprises from about 10 mg to about 100 mg rifaximin.

In any of the foregoing embodiments, the subject can be administered the composition about 24 hours to 7 or more days prior to administration of the substrate and/or PET scan. In some embodiments, the subject is administered the composition about 48 hours to 5 days prior to administration of the substrate and/or PET scan. In some embodiments, the subject is administered the composition about 48 hours prior to administration of the substrate and/or PET scan. In some embodiments, the subject is administered the composition about 5 days prior to administration of the substrate and/or PET scan.

In any of the foregoing embodiments, rifaximin can include one or more of rifaximin or a Form α, Form β, Form γ, Form δ, Form ε, Form ξ, Form η, Form ι, Form kappa, Form lambda, Form mu, From omicron, Form pi, Form theta, Form xi, mesylate Form or amorphous Forms of rifaximin and a pharmaceutically acceptable carrier. The rifaximin may be formulated as a pharmaceutical composition. In some embodiments, the pharmaceutical composition further comprises excipients.

In some embodiments, the excipients comprise one or more of a diluting agent, binding agent, lubricating agent, disintegrating agent, coloring agent, flavorings agent or sweetening agent.

In some embodiments, the composition is formulated for selected coated and uncoated tablets, hard and soft gelatin capsules, sugar-coated pills, lozenges, wafer sheets, pellets and powders in sealed packet.

Other embodiments are disclosed infra.

### BRIEF DESCRIPTION OF THE DRAWINGS

FIG. 1 illustrates a graph (A) and a statistical scatter plot (B) that show the results of maximum FDG uptake (SUVMax) in the cecum of rifaximin-treated patients prior to and after their scans.

FIG. 2 is a graph that shows the results of maximum FDG uptake (SUVMax) in the cecum of rifaximin-treated patients whose prior scan indicated high FDG uptake (SUVMax >2) prior to and after their scans.

FIG. 3 is a statistical scatter plot graph that shows normalized average FDG uptake (normalized cecal SUVAvg) in rifaximin-treated patients prior to and after their scans.

FIG. 4 is a bar chart that shows the frequency of overall colonic FDG (SUV) intensity grade in untreated (control) and rifaximin-treated patients.

FIG. 5 is a graph (A) and a statistical scatter plot (B) that show the results of maximum FDG uptake (SUVMax) in the cecum of control patients prior to and after their scans.

FIG. 6 are scatter plots that show maximum FDG uptake (SUVMax) in the cecum of rifaximin-treated patient scans and random control scans.

FIG. 7 is a plot showing overall colonic SUV grade vs. cecal SUVmax level for all scans scored in the study described herein (n=120).

### DETAILED DESCRIPTION

PET-CT scans are used in oncology for diagnostic and treatment purposes. Such scans are provided by administering a substrate, FDG, that is taken up or absorbed by tissues that metabolize glucose for rapid growth, such as tumors. For assessment of intestinal disease, the utility of FDG-PET is limited by localized physiological FDG uptake. However, up to 20% of PET-CT scans exhibit high FDG uptake in the healthy intestinal tract. The cellular location of the FDG in the colon is unknown, but FDG uptake in the colon is located within the intestinal lumen, which contains high concentrations of commensal bacteria. Without wishing to be bound by theory, it is hypothesized that intestinal bacteria can concentrate luminal FDG, leading to false positive FDG uptake signals from the intestinal tract. Accordingly, embodiments are directed to administration of antibiotics prior to administration of PET-CT scans to reduce false positive substrate uptake activity and to improve interpretation of PET-CT images.

In some embodiments, the administered antibiotic is rifaximin. Rifaximin (USAN, INN; see The Merck Index, XIII Ed., 8304, CAS No. 80621-81-4), (2S,16Z,18E,20S,21S,22R, 23R,24R,25S,26S,27S,28E)-5,6,21,23,25 Pentahydroxy -27-methoxy-2,4,11,16,20,22,24,26-octamethyl-2,7-(epoxypentadeca-(1,11,13)trienimino)benzofuro(4,5-e)pyrido(1,2,-a)benzimidazole-1,15(2H)-dione,25-acetate), is a semi-synthetic antibiotic produced from rifamycin O. Rifaximin is a molecule belonging to the rifamycin class of antibiotics, e.g., a pyrido-imidazo rifamycin. Rifaximin exerts a broad antibacterial activity, for example, in the gastrointestinal tract against localized gastrointestinal bacteria that cause infectious diarrhea, irritable bowel syndrome, small intestinal bacterial overgrowth, Crohn's disease, and/or pancreatic insufficiency.

Rifaximin is also described in Italian Patent IT 1154655 and EP 0161534. EP patent 0161534 discloses a process for rifaximin production using rifamycin O as the starting material (The Merck Index, XIII Ed., 8301). U.S. Pat. No. 7,045,620 B1 discloses polymorphic forms of rifaximin, as do U.S. Ser. No. 11/658,702; U.S. Ser. No. 61/031,329; U.S. Ser. No. 12/119,622; U.S. Ser. No. 12/119,630; U.S. Ser. No. 12/119,612; U.S. Ser. No. 12/119,600; U.S. Ser. No. 11/873,841; Publication WO 2006/094662; and U.S. Ser. No. 12/393012. The applications and patents referred to here are incorporated herein by reference in their entirety for all purposes.

“Rifaximin”, as used herein, includes solvates and polymorphous forms of the molecule, including, for example, Form α, Form β, Form γ Form δ, Form ε, Form ξ, Form η, Form ι, Form kappa, Form theta, From mu, From omicron, Form pi, Form lambda, Form xi, mesylate Form or amorphous Forms of rifaximin. These forms are described in more detail, for example, in EP 05 004 635.2, filed 3 Mar. 2005; U.S. Pat. No. 7,045,620; U.S. Pat. No. 7,612,199; U.S. Pat. No. 7,709,634; U.S. Pat. No. 7,915,275; U.S. Pat. No. 8,067,429; U.S. Pat. No. 8,193,196; U.S. Pat. No. 8,227,482; G. C. Viscomi, et al., CrystEngComm, 2008, 10, 1074-1081 (April 2008), US Patent Publication No. 2010/0174064, US Patent Publication No. 2009/0028940, US Patent Publication No. 2005/0272754 and U.S. Patent Publication No. 2012/0108620. Each of these references is hereby incorporated by reference in entirety.

Medicinal preparations may contain rifaximin together with usual excipients, discussed infra.

“Polymorphs” or “polymorphic forms” as used herein, refer to the occurrence of different crystalline forms of a single compound in distinct hydrate status, e.g., a property of some compounds and complexes. Thus, polymorphs are distinct solids sharing the same molecular formula, yet each polymorph may have distinct physical properties. Therefore, a single compound may give rise to a variety of polymorphic forms where each form has different and distinct physical properties, such as solubility profiles, melting point temperatures, hygroscopicity, particle shape, density, flowability, compatibility and/or x-ray diffraction peaks. The solubility of each polymorph may vary, thus, identifying the existence of pharmaceutical polymorphs is essential for providing pharmaceuticals with predictable solubility profiles. It is desirable to investigate all solid state forms of a drug, including all polymorphic forms, and to determine the stability, dissolution and flow properties of each polymorphic form. Polymorphic forms of a compound can be distinguished in a laboratory by X-ray diffraction spectroscopy and by other methods such as, infrared spectrometry. For a general review of polymorphs and the pharmaceutical applications of polymorphs see G. M. Wall, Pharm Manuf. 3, 33 (1986); J. K. Haleblian and W. McCrone, J Pharm. Sci., 58, 911 (1969); and J. K. Haleblian, J. Pharm. Sci., 64, 1269 (1975), all of which are incorporated herein by reference. As used herein, the term polymorph is occasionally used as a general term in reference to the forms of rifaximin and include within the context, salt, hydrate, polymorph and amorphous forms of rifaximin disclosed herein. This use depends on context and will be clear to one of skill in the art. Exemplary polymorphic forms of rifaximin useful in the methods and kits described herein are set forth in the published patent applications set forth above.

The term “effective amount” includes an amount effective, at dosages and for periods of time necessary, to achieve the desired result, e.g., sufficient to decrease, prevent or inhibit uptake of a substrate by intestinal bacteria as described herein. An effective amount is also one in which any toxic or detrimental effects (e.g., side effects) of rifaximin are outweighed by the therapeutically beneficial effects.

As used herein, “subject” includes organisms which are capable of undergoing a PET-CT scan as described herein or who could otherwise benefit from the administration of rifaximin as described herein, such as human and non-human animals. Preferred human animals include human subjects. The term “non-human animals” includes all vertebrates, e.g., mammals, e.g., rodents, e.g., mice, and non-mammals, such as non-human primates, e.g., sheep, dog, cow, chickens, amphibians, reptiles, etc.

The term “administration” or “administering” includes routes of introducing rifaximin to a subject to perform its intended function. Examples of routes of administration that may be used include injection, oral, rectal and transdermal. The pharmaceutical preparations may be given by forms suitable for each administration route. For example, these preparations are administered in tablets or capsule form, by injection, ointment, suppository, etc. administration by injection or infusion; topical by lotion or ointment; and rectal by suppositories. Oral administration is preferred. The injection can be bolus or can be continuous infusion. Depending on the route of administration, the rifaximin can be coated with or disposed in a selected material to protect it from natural conditions that may detrimentally affect its ability to perform its intended function. Rifaximin can be administered alone, or in conjunction with either another agent or agents as described above or with a pharmaceutically-acceptable carrier, or both. Rifaximin can be administered prior to the administration of the other agent, simultaneously with the agent, or after the administration of the agent.

Administration “in combination with” one or more further therapeutic agents includes simultaneous (concurrent) and consecutive administration in any order.

As will be readily apparent to one skilled in the art, the useful in vivo dosage to be administered and the particular mode of administration will vary depending upon the age, weight and mammalian species treated, the particular compounds employed, and/or the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved.

The term “obtaining” as in “obtaining rifaximin” is intended to include purchasing, synthesizing or otherwise acquiring rifaximin. For example, obtaining rifaximin can include purchasing, synthesizing or otherwise acquiring rifaximin.

The term “pharmaceutical agent composition” (or agent or drug) as used herein refers to a chemical compound, composition, agent or drug capable of inducing a desired therapeutic effect when properly administered to a patient. It does not necessarily require more than one type of ingredient.

Provided herein are methods of reducing the uptake of a substrate by non-metastatic cells comprising administering to a subject in need thereof an effective amount of rifaximin. In some embodiments, the substrate uptake by non-metastatic cells is reduced in the subject relative to a subject that has not been administered rifaximin. For example, the substrate uptake can be reduced by between about 2% to about 65%, or any percentage in between. In some embodiments, the substrate uptake is reduced by about 2%, 5%, 10%, 15%, 20%, 25%, 30%, 35%, 40%, 45%, 50%, 55%, 60% or 65%.

Also provided herein are methods of preventing or inhibiting the uptake a substrate by non-metastatic cells comprising administering to a subject in need thereof an effective amount of rifaximin.

In some embodiments, the non-metastatic cells are located in the intestinal tract of the subject. Non-metastatic cells include, for example, metabolically active bacteria. In some embodiments, the non-metastatic cells are intestinal bacteria. In some embodiments, the non-metastatic cells are colonic bacteria. For example, the metabolically active bacteria can include, but are not limited to, E. coli.

Also provided herein are methods of reducing background noise of a PET-CT scan, comprising administering to a subject in need thereof an effective amount of rifaximin. In some embodiments, the background noise is reduced in the subject relative to that of a subject that has not been administered rifaximin. For example, the background noise can be reduced by between about 2% to about 65%, or any percentage in between. In some embodiments, the background noise is reduced by about 2%, 5%, 10%, 15%, 20%, 25%, 30%, 35%, 40%, 45%, 50%, 55%, 60% or 65%.

Embodiments are also directed to methods of reducing the risk of a false positive diagnosis in a subject undergoing a PET-CT scan, comprising administering to the subject an effective amount of rifaximin, wherein administration of rifaximin results in the reduction in the risk of a false positive diagnosis relative to that of a subject which was not administered rifaximin prior to a PET-CT scan.

In some embodiments, the subject is undergoing a PET-CT scan to diagnose a condition, disease or symptom associated with a gastrointestinal tract disorder. For example, the subject can undergo a PET-CT scan to diagnose a cancer of the gastrointestinal tract or inflammation of the gastrointestinal tract.

In some embodiments, the subject is undergoing a PET-CT scan to monitor a condition, disease or symptom associated with a gastrointestinal tract disorder, or to monitor a therapy that is being administered to treat the condition, disease or symptom associated with a gastrointestinal tract disorder. The condition, disease or symptom can be, for example, a cancer of the gastrointestinal tract of inflammation of the gastrointestinal tract.

The substrate can be any radiolabeled sugar analog that is taken up by metabolically-active cells. For example, the substrate can be glucose or sucrose that is modified with a radiolabel to become a radioactive sugar. The radiolabel can be an isotope with a short half-life, such as, for example, ¹¹C, ¹³N, ¹⁵O, ¹⁸F, ⁸²Rb, or any combination thereof. In some embodiments, the substrate is 2-deoxy-2-[¹⁸F]fluoro-D-glucose, also known as FDG.

Rifaximin may be used in various regimens. For example, rifaximin can be administered, for example, twice a day, three times a day, or four times or more often as necessary per day. Rifaximin may be administered in doses, for example of from about 10 mg once daily to about 3000 mg TID. For example, rifaximin can be administered in daily doses of about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, In some embodiments, rifaximin can be administered in daily doses of about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg. In some embodiments, rifaximin can be administered in daily doses of about 550 mg, about 600 mg, about 650 mg, about 700 mg, about 750 mg, about 800 mg, about 850 mg, about 900 mg, about 950 mg, or about 1000 mg. In some embodiments, rifaximin can be administered in daily doses of about 1100 mg about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, or about 3000 mg, In some embodiments, rifaximin can be administered in doses of about 10 mg BID, about 15 mg BID, about 20 mg BID, about 25 mg BID, about 30 mg BID, about 35 mg BID, about 40 mg BID, about 45 mg BID, about 50 mg BID, about 55 mg BID, about 60 mg BID, about 65 mg BID, about 70 mg BID, about 75 mg BID, about 80 mg BID, about 85 mg BID, about 90 mg BID, about 95 mg BID, or about 100 mg BID. In some embodiments, rifaximin can be administered in doses of about 125 mg BID, about 150 mg BID, about 175 mg BID, about 200 mg BID, about 225 mg BID, about 250 mg BID, about 275 mg BID, about 300 mg BID, about 325 mg BID, about 350 mg BID, about 375 mg BID, about 400 mg BID, about 425 mg BID, about 450 mg BID, about 475 mg BID, or about 500 mg BID. In some embodiments, rifaximin can be administered in doses of about 550 mg BID, about 600 mg BID, about 650 mg BID, about 700 mg BID, about 750 mg BID, about 800 mg BID, about 850 mg BID, about 900 mg BID, about 950 mg BID, or about 1000 mg BID. In some embodiments, rifaximin can be administered in doses of about 1100 mg BID, about 1200 mg BID, about 1300 mg BID, about 1400 mg BID, about 1500 mg BID, about 1600 mg BID, about 1700 mg BID, about 1800 mg BID, about 1900 mg BID, about 2000 mg BID, about 2100 mg BID, about 2200 mg BID, about 2300 mg BID, about 2400 mg BID, about 2500 mg BID, about 2600 mg BID, about 2700 mg BID, about 2800 mg BID, about 2900 mg BID or about 3000 mg BID, In some embodiments, rifaximin can be administered in doses of about 25 mg TID, about 30 mg TID, about 35 mg TID, about 40 mg TID, about 45 mg TID, about 50 mg TID, about 55 mg TID, about 60 mg TID, about 65 mg TID, about 70 mg TID, about 75 mg TID, about 80 mg TID, about 85 mg TID, about 90 mg TID, about 95 mg TID, or about 100 mg TID. In some embodiments, rifaximin can be administered in doses of about 125 mg TID, about 150 mg TID, about 175 mg TID, about 200 mg TID, about 225 mg TID, about 250 mg TID, about 275 mg TID, about 300 mg TID, about 325 mg TID, about 350 mg TID, about 375 mg TID, about 400 mg TID, about 425 mg TID, about 450 mg TID, about 475 mg TID, or about 500 mg TID, In some embodiments, rifaximin can be administered in doses of about 550 mg TID, about 600 mg TID, about 650 mg TID, about 700 mg TID, about 750 mg TID, about 800 mg TID, about 850 mg TID, about 900 mg TID, about 950 mg TID, or about 1000 mg TID. In some embodiments, rifaximin can be administered in doses of about 1100 mg TID, about 1200 mg TID, about 1300 mg TID, about 1400 mg TID, about 1500 mg TID, about 1600 mg TID, about 1700 mg TID, about 1800 mg TID, about 1900 mg TID, about 2000 mg TID, about 2100 mg TID, about 2200 mg TID, about 2300 mg TID, about 2400 mg TID, about 2500 mg TID, about 2600 mg TID, about 2700 mg TID, about 2800 mg TID, about 2900 mg TID or about 3000 mg TID. The rifaximin may be administered, for example, in tablet form, powdered form, liquid form or in capsules. In some embodiments, rifaximin can be administered in a time-released formulation.

In some embodiments, rifaximin is administered as a solid dispersion. For example, rifaximin can be administered as a solid dispersion containing from about 5 to 550 mg of rifaximin. In some embodiments, rifaximin can be administered as a solid dispersion containing from about 10 to 100 mg of rifaximin. Exemplary solid dispersions of rifaximin are described in WO 2012/009388, which is incorporated herein by reference in its entirety.

In some embodiments, the rifaximin is administered to a subject from between about 12 hours to 2 weeks prior to administration of the PET-CT scan. In some embodiments, rifaximin is administered for between about 24 hours to 10 days prior to administration of the PET-CT scan. For example, rifaximin can be administered about 24 hours, 48 hours, 72 hours, 96 hours, 5 days, 6, days, 7, days, 8 days, 9 days or 10 days prior to administration of the PET-CT scan. In some embodiments, rifaximin is administered for between about 24 hours to 7 days prior to administration of the PET-CT scan. In some embodiments, rifaximin is administered for between about 48 hours to 5 days prior to administration of the PET-CT scan. In some embodiments, rifaximin is administered for between about 24 hours to 72 hours prior to administration of the PET-CT scan. The rifaximin may be administered intermittently or continuously during the course of treatment, or as a dosing regimen as described herein.

In some embodiments, rifaximin may be administered in combination with the substrate. In some embodiments, rifaximin is administered prior to administration of the substrate. For example, rifaximin can be administered to a subject from between about 12 hours to 2 weeks prior to administration of the substrate. In some embodiments, rifaximin is administered for between about 24 hours to 10 days prior to administration of the substrate. For example, rifaximin can be administered about 24 hours, 48 hours, 72 hours, 96 hours, 5 days, 6, days, 7, days, 8 days, 9 days or 10 days prior to administration of the substrate. The rifaximin may be administered intermittently or continuously during the course of treatment, or as a dosing regimen as described herein.

Other appropriate dosages for the methods as disclosed herein may be determined by health care professionals or by the subject. The amount of rifaximin administered daily may be increased or decreased based on the weight, age, health, sex or medical condition of the subject. One of skill in the art would be able to determine the proper dose for a subject based on this disclosure.

Embodiments also relate to pharmaceutical compositions, comprising an effective amount of a rifaximin as described herein and a pharmaceutically acceptable carrier.

In some embodiments, the pharmaceutical composition comprises rifaximin or any polymorphic form thereof and a pharmaceutically acceptable carrier. That is, formulations may contain only one polymorph or may contain a mixture of more than one polymorph. Polymorph, in this context, refers to any physical form, hydrate, acid, salt or the like of rifaximin. Mixtures may be selected, for example on the basis of desired amounts of systemic adsorption, dissolution profile, desired location in the digestive tract to be treated, and the like. The pharmaceutical composition further comprises excipients, for example, one or more of a diluting agent, binding agent, lubricating agent, disintegrating agent, coloring agent, flavoring agent or sweetening agent. Compositions may be formulated for selected coated and uncoated tablets, hard and soft gelatin capsules, sugar-coated pills, lozenges, wafer sheets, pellets and powders in sealed packet.

In some embodiments, rifaximin is administered to the subject using a pharmaceutically-acceptable formulation, e.g., a pharmaceutically-acceptable formulation that provides sustained delivery of the rifaximin to a subject for at least 12 hours, 24 hours, 36 hours, 48 hours, 96 hours, one week or two weeks after the pharmaceutically-acceptable formulation is administered to the subject.

In some embodiments, these pharmaceutical compositions are suitable for topical or oral administration to a subject. In other embodiments, as described in detail below, the pharmaceutical compositions described herein may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) intrarectally, for example, as a pessary, cream or foam; or (4) aerosol, for example, as an aqueous aerosol, liposomal preparation or solid particles containing the compound.

The phrase “pharmaceutically acceptable” refers to those rifaximin compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing rifaximin include those suitable for oral, rectal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1% to about 99% of active ingredient, preferably from about 5% to about 70%, most preferably from about 10% to about 30%.

Liquid dosage forms for oral or rectal administration of rifaximin include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to the rifaximin, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions for rectal administration may be presented as a suppository, which may be prepared by mixing rifaximin with one or more suitable nonirritating excipients or carriers comprising, for example, cocoa butter, polyethylene glycol, a suppository wax or a salicylate, and which is solid at room temperature, but liquid at body temperature and, therefore, will melt in the rectum cavity and release the active agent.

Examples of suitable aqueous and non-aqueous carriers which may be employed in the pharmaceutical compositions can include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, to prolong the effect of a drug, it is desirable to alter the absorption of the drug. This may be accomplished by the use of a liquid suspension of crystalline, salt or amorphous material having poor water solubility. The rate of absorption of the drug may then depend on its rate of dissolution which, in turn, may depend on crystal size and crystalline form. Alternatively, delayed absorption of a drug form is accomplished by dissolving or suspending the drug in an oil vehicle.

When the rifaximin is administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, rifaximin, which may be used in a suitable hydrated form, and/or the pharmaceutical compositions, are formulated into pharmaceutically-acceptable dosage forms by conventional methods known to those of skill in the art.

Actual dosage levels and time course of administration of the active ingredients in the pharmaceutical compositions may be varied so as to obtain an amount of the active ingredient which is effective to achieve the desired therapeutic response for a particular patient, composition, and mode of administration, without being toxic to the patient. An exemplary dose range is from 25 to 3000 mg per day.

In combination therapy treatment, both the compounds and the other drug agent(s) are administered to mammals (e.g., humans, male or female) by conventional methods. The agents may be administered in a single dosage form or in separate dosage forms. Effective amounts of the other agents are well known to those skilled in the art. However, it is well within the skilled artisan's purview to determine the other therapeutic agent's optimal effective-amount range. In one embodiment in which another therapeutic agent is administered to an animal, the effective amount of the compound is less than its effective amount in case the other therapeutic agent is not administered. In another embodiment, the effective amount of the conventional agent is less than its effective amount in case the compound is not administered. In this way, undesired side effects associated with high doses of either agent may be minimized. Other potential advantages (including without limitation improved dosing regimens and/or reduced drug cost) will be apparent to those skilled in the art.

In some embodiments, rifaximin and the other agent are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In some embodiments, rifaximin and the other agent are administered within the same patient's visit.

Kits are also provided herein, for example, kits for reducing, preventing or inhibiting the uptake of a substrate by non-metastatic cells. The kits may contain, for example, a polymorph or amorphous form of rifaximin and instructions for use. The instructions for use may contain prescribing information, dosage information, storage information, and the like.

Kits are also provided herein, for example, kits for reducing the background imaging noise of a PET scan for a subject. The kits may contain, for example, a polymorph or amorphous form of rifaximin and instructions for use. The instructions for use may contain prescribing information, dosage information, storage information, and the like.

Kits are also provided herein, for example, kits for reducing the risk of false positive diagnosis in a subject who will undergo a PET scan. The kits may contain, for example, a polymorph or amorphous form of rifaximin and instructions for use. The instructions for use may contain prescribing information, dosage information, storage information, and the like.

In some embodiments, the label describes adverse events comprising one or more of infections and infestations, gastrointestinal disorders, nervous system disorders, and musculoskeletal and connective tissue disorders.

Label instructions can include, for example, instructions to take the rifaximin for between about 12 hours to 2 weeks prior to administration of the substrate or PET scan. The instructions could also read, for example, to take about 1100 mg/day or 1650 mg/day of rifaximin for between about 12 hours to 2 weeks prior to administration of the substrate or PET scan.

Packaged compositions are also provided, and may comprise a therapeutically effective amount of one or more of a one or more of Form α, Form β, Form γ Form δ, Form ε, Form ξ, Form η, Form ι, Form kappa, Form theta, From mu, From omicron, Form pi, Form lambda, Form xi, mesylate Form or amorphous Forms of rifaximin and a pharmaceutically acceptable carrier or diluent, wherein the composition is formulated for administration to a subject who will undergo a PET scan.

In some embodiments, the packaged composition can comprise rifaximin as a solid dispersion. For example, rifaximin can be administered as a solid dispersion containing from about 5 to 550 mg of rifaximin. In some embodiments, rifaximin can be administered as a solid dispersion containing from about 10 to 100 mg of rifaximin. Solid dispersions of rifaximin are described, for example, in WO 2012/009388, which is incorporated herein by reference in its entirety.

### EXAMPLES

It should be appreciated that embodiments should not be construed to be limited to the example, which is now described; rather, embodiments should be construed to include any and all applications provided herein and all equivalent variations within the skill of the ordinary artisan.

### Example 1

This example relates to a study to determine whether an oral antibiotic, rifaximin, administered prior to PET-CT scan can reduce physiologic FDG uptake in the intestinal tract. Thirty (30) patients scheduled for clinically-indicated PET-CT scans were enrolled to receive rifaximin 550 mg twice daily (BID) for two days prior to their scans. Each patient had their prior scheduled PET-CT used as control; that is, their prior PET-CT scan was evaluated as a pre-treatment baseline for intestinal FDG uptake (paired controls). The PET-CT scans of a randomly-selected cohort of 90 patients also scheduled for clinically-indicated PET-CT during the same time period were also used as controls (independent controls). All images were stripped of identifying data, and SUVMax (maximum-pixel-value standardized uptake value) and SUVAvg (average standardized uptake value) for all scans were measured by a radiologist blinded to treatment/control status and normalized to liver SUV, which was selected as the reference for normal tissue activity. A qualitative score of colonic uptake (0-4) was also assessed. Statistical analysis was performed by comparing mean SUV cecum/liver by paired t-test (pre & post) or unpaired t-test (treated & controls).

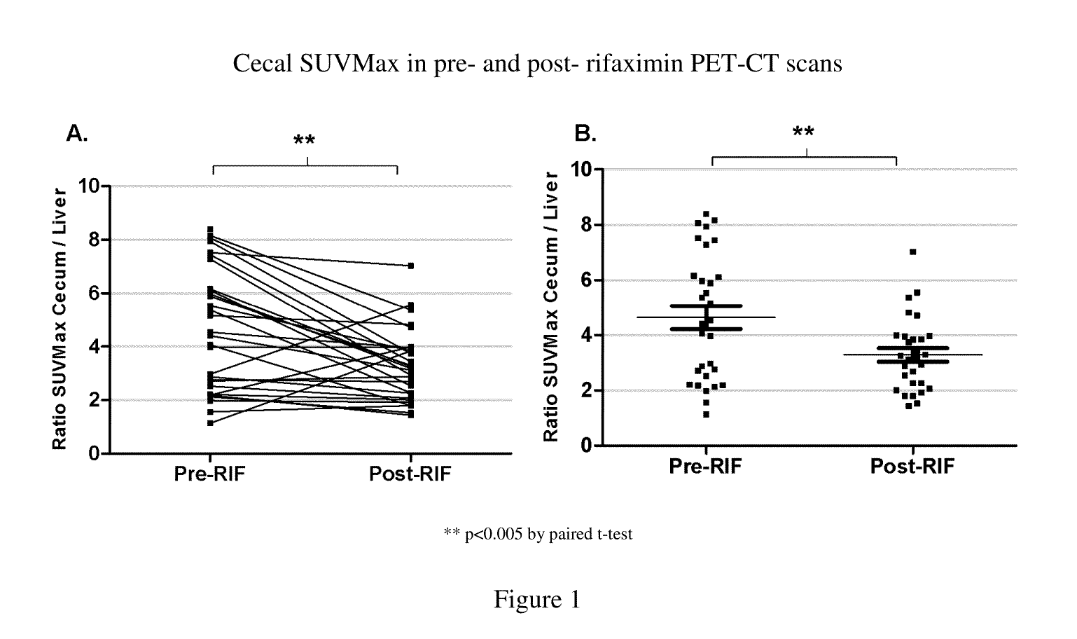

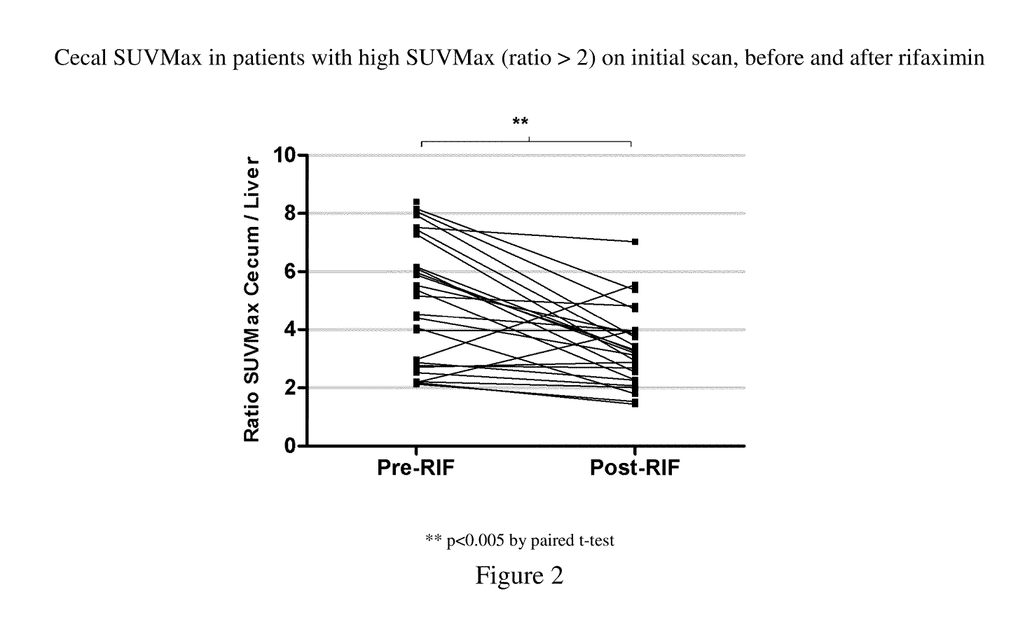

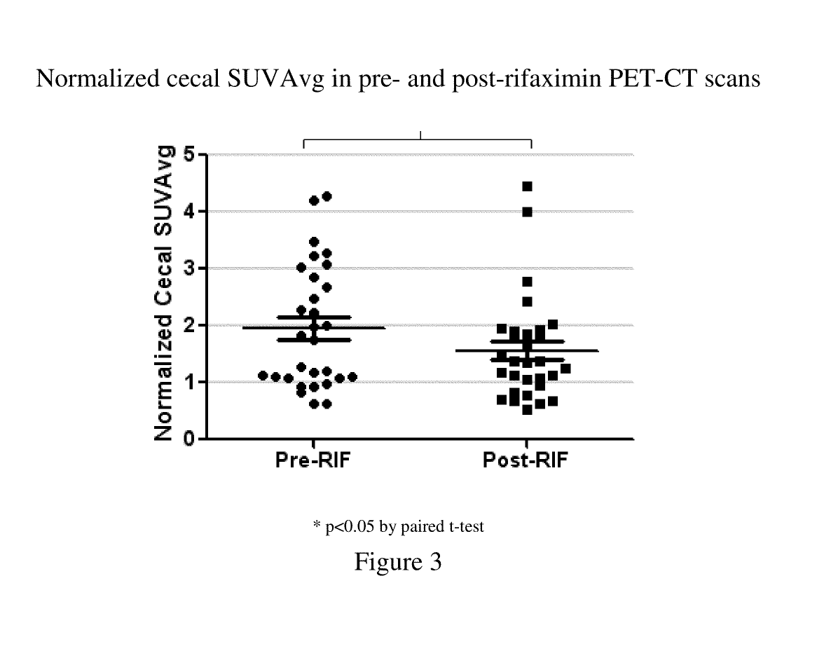

The mean cecal SUVMax was 4.6 (SD=2.3) without rifaximin, and 3.5 (SD=1.6) with rifaximin (p=0.002 by paired t-test, FIG. 1). This corresponded to a 26% difference in cecal SUV max between pre- and post-rifaximin images. In those patients whose SUVMax was >2 in their prior scan, their mean cecal SUVMax was 5.0 without rifaximin, and 3.4 with rifaximin (p=0.002 by paired t-test, FIG. 2). Normalized cecal SUVAvg was 2.0 (SD=1) without rifaximin, and 1.6 with rifaximin (SD=1) (p=0.02 by paired t-test, FIG. 3).

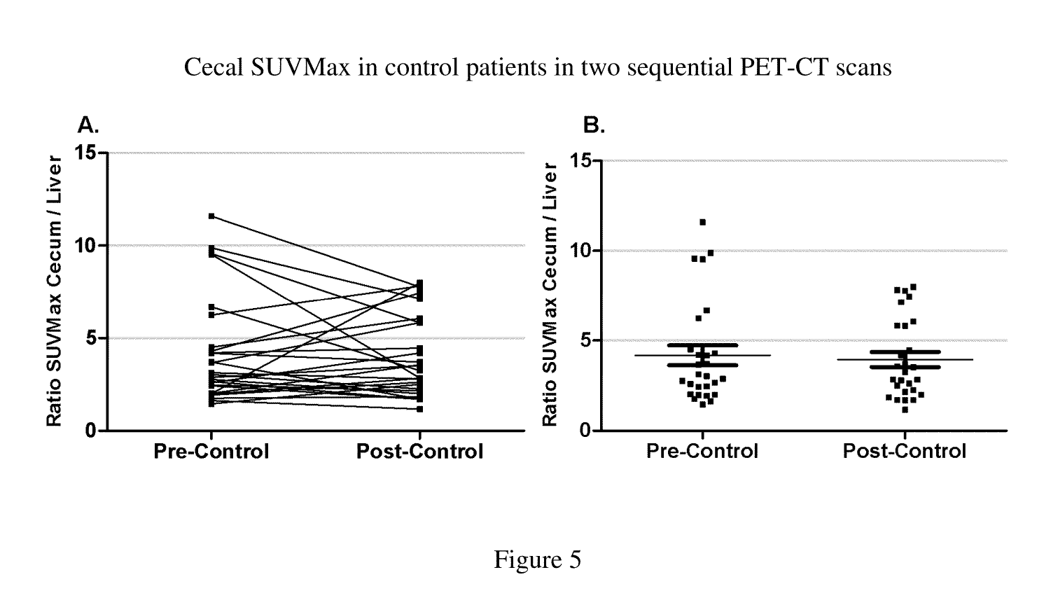

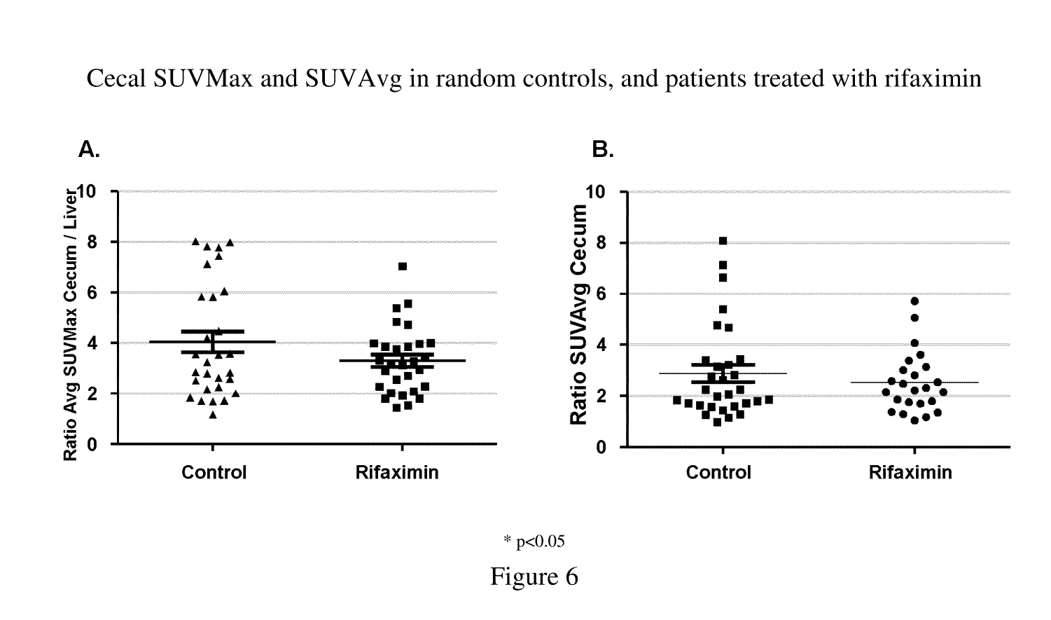

These differences were not seen when 2 sequential scans were compared from a randomly-selected cohort of non-rifaximin patients (FIG. 5) nor in sequential scans in in the 30 non-rifaximin patients (Table 1). There was good correlation in SUVMax interpretation between two blinded readers, independently reading each scan (r²=0.8). There was no significant difference in cecal SUVMax between rifaximin-treated patients (mean 3.4) and random control scans (mean 4.2, p=0.2 by unpaired t-test, FIG. 6).

Since images with high SUV signals are more likely to lead to “false positive” results, the effect of rifaximin was also analyzed in those patients (n=27) that had a cecal SUVmax/liver ratio >2 in their pre-rifaximin scans. In this group, 21/27 (78%) exhibited a lower cecal SUVmax/liver ratio after rifaximin, 4/27 (14%) had no major change, and 2/27 (7%) had a higher ratio post-rifaximin (p=0.0005 by paired t-test of absolute levels). In the control group, only 9/25 (36%) had a lower level, 5/27 (20%) were unchanged, and 11/25 (44%) had a higher cecal SUVmax/liver ratio in sequential scans (p=0.2 by paired t-test).

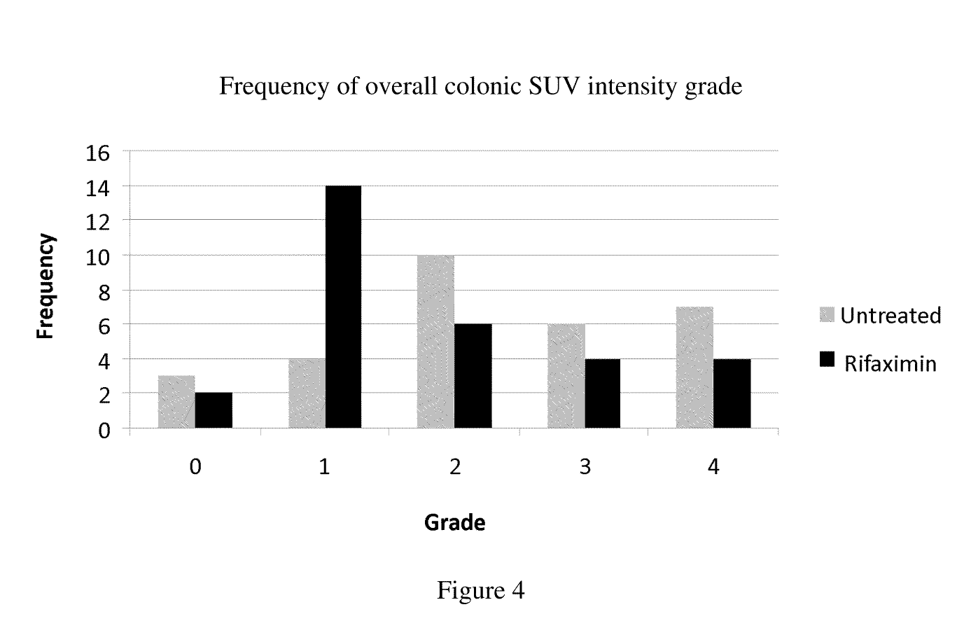

Although the cecum is the region of the colon with the highest bacterial concentrations, we also investigated the effect of rifaximin on a global measure of colonic SUV. Each scan was given a score of 0-4 on overall colonic SUV by a blinded reader. As can be seen in FIG. 4, there was a left-shift in the distribution of colonic SUV grades from higher to lower grades in the patients' post-rifaximin scans. When scores were dichotomized (≦1 or >1), 53% of scans in patients treated with rifaximin had scores <1, compared with 23% of scans without rifaximin (OR for score <1 3.8, 95% CI 1.2, 11.4, p=0.02 by χ² test). In contrast, there was no significant difference in the proportions of scans with scores <1 between sequential control scans (48% vs 30%, p=0.1 by χ² test). The overall percentage of patients with grade of colonic FDG intensity >1 was lower with rifaximin, than without rifaximin (FIG. 4).

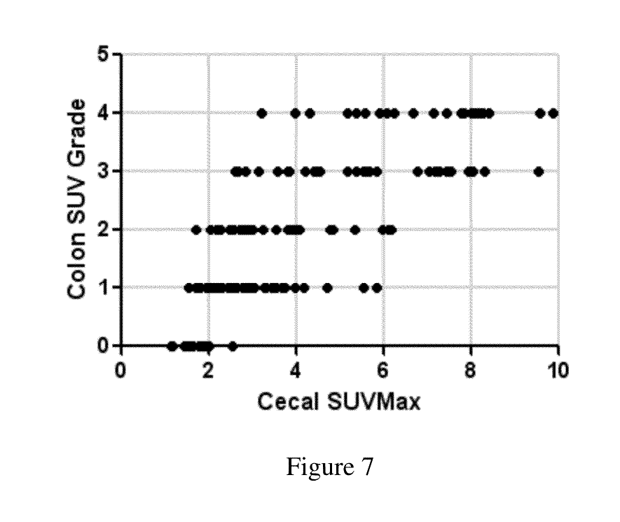

To determine if the overall colonic grade correlated with cecal SUVmax scores, the overall colonic grade of each scan was plotted against its corresponding cecal SUVmax score. As can be seen from FIG. 7, there was good correlation between these scores, suggesting the overall alterations in a global interpretation of SUV reflected the more precise measurements of cecal SUVmax (Spearman r²=0.8).

The results show a 26% reduction of FDG activity in the cecum of patients treated with rifaximin, when compared to their untreated pre-rifaximin scans (SUVmax, p=0.002). Controls exhibited no significant difference in the same measurements applied to sequential scans (−6%, p=0.23). The qualitative score of whole colonic uptake was also reduced in the rifaximin pre-treated group from a mean of 2.3 to 1.8 (p=0.07). No adverse events were reported in the rifaximin-treated group. Table 1 provides a summary of the results.

TABLE 1
SUV measurements in pre- and post-rifaximin scans (30
patients treated with rifaximin, 30 control patients)

| Measure | Pre-RIF | Post-RIF | p |
|---|---|---|---|
| Mean cecal SUVmax | 4.6 | 3.4 | 0.002 |
| Mean cecal SUVavg | 2.0 | 1.6 | 0.02 |
| Mean colon SUVmax | 4.0 | 3.8 | 0.7 |
| Measure | Pre-Control | Post-Control | p |
| Mean cecal SUVmax | 4.3 | 4 | 0.6 |
| Mean cecal SUVavg | 1.6 | 1.8 | 0.2 |
| Mean colon SUVmax | 3.9 | 4 | 0.7 |

In conclusion, patients who received rifaximin for 2 days prior to PET-CT scans exhibited a significant reduction in FDG uptake when compared to their pre-rifaximin scan. In particular, patients with high baseline cecal SUVmax levels exhibited significantly lower levels after 2 days of rifaximin pre-treatment. The dominant effect on overall colonic SUV signal appeared to be a “down-grading” of FDG signal intensity (FIG. 4), which occurred in almost all (80%) of patients with an initial high cecal SUVMax. This effect was not seen in sequential scans in control patients.

Accordingly, pre-treatment with rifaximin can reduce physiological F-18 uptake in the intestinal tract. Rifaximin has clinical utility in reducing false positive FDG uptake in colonic segments on PET-CT scans. A longer duration of therapy may be useful in enhancing the effects on colonic FDG uptake, and further studies can be carried out to optimize the antibiotic regiment for minimizing false-positive FDG activity in the normal intestinal lumen.

Embodiments are directed to reducing uptake of a substrate by metabolically active cells in a subject, comprising administering to the subject a composition comprising rifaximin. In some embodiments, the metabolically active cells are non-metastatic cells such as intestinal bacteria and colonic bacteria. In some embodiments, the metabolically active cells include, for example, E. coli. In some embodiments, the subject is administered 1100 mg of rifaximin per day. In some embodiments, the subject is administered 550 mg rifaximin twice per day. In some embodiments, the composition comprising rifaximin is administered to the subject about 48 hours prior to administration of the substrate. In some embodiments, the composition comprising rifaximin is administered to the subject at least about 48 hours prior to administration of the substrate. In some embodiments, the substrate is FDG.

Embodiments are also directed to reducing the background false positive noise of a PET scan of a subject, comprising administering to the subject a composition comprising rifaximin. In some embodiments, the subject is administered 1100 mg of rifaximin per day. In some embodiments, the subject is administered 550 mg rifaximin twice per day. In some embodiments, the composition comprising rifaximin is administered to the subject about 48 hours prior to administration of the substrate. In some embodiments, the composition comprising rifaximin is administered to the subject at least about 48 hours prior to administration of the substrate. In some embodiments, the background noise is reduced by about 20% to 35%.

### Example 2

Patients scheduled for clinically-indicated PET-CT scan are administered rifaximin 550 mg twice daily (BID) for five days prior to their scans. Each patient has their prior scheduled PET-CT used as control; that is, their prior PET-CT scan is evaluated as a pre-treatment baseline for intestinal FDG uptake (paired controls). The PET-CT scans of a randomly-selected cohort of patients also scheduled for clinically-indicated PET-CT during the same time period are used as independent controls. Cecal SUVMax (maximum-pixel-value standardized uptake value) and SUVAvg (average standardized uptake value) for all scans are measured by a radiologist blinded to treatment/control status and normalized to liver SUV, which is selected as the reference for normal tissue activity. A qualitative score of colonic uptake (0-4) is also assessed. Statistical analysis was performed by comparing mean SUV cecum/liver by paired t-test (pre & post) or unpaired t-test (treated & controls). The effect of rifaximin on a global measure of colonic SUV is also measured.

Patients who receive rifaximin for five days prior to PET-CT scans exhibit a significant reduction in FDG uptake when compared to their pre-rifaximin scan. Accordingly, pre-treatment with rifaximin for five days prior to PET-CT scans can reduce physiological F-18 uptake in the intestinal tract.

Embodiments are directed to reducing uptake of a substrate by metabolically active cells in a subject, comprising administering to the subject a composition comprising rifaximin. In some embodiments, the metabolically active cells are non-metastatic cells such as intestinal bacteria and colonic bacteria. In some embodiments, the metabolically active cells include, for example, E. coli. In some embodiments, the subject is administered 1100 mg of rifaximin per day. In some embodiments, the subject is administered 550 mg rifaximin twice per day. In some embodiments, the composition comprising rifaximin is administered to the subject about five days prior to administration of the substrate. In some embodiments, the composition comprising rifaximin is administered to the subject at least about five days prior to administration of the substrate. In some embodiments, the substrate is FDG.

Embodiments are also directed to reducing the background false positive noise of a PET scan of a subject, comprising administering to the subject a composition comprising rifaximin. In some embodiments, the subject is administered 1100 mg of rifaximin per day. In some embodiments, the subject is administered 550 mg rifaximin twice per day. In some embodiments, the composition comprising rifaximin is administered to the subject about five days prior to administration of the substrate. In some embodiments, the composition comprising rifaximin is administered to the subject at least about five days prior to administration of the substrate. In some embodiments, the background noise is reduced by about 10% to 50%. In some embodiments, the background noise is reduced by about 15% to 40%. In some embodiments, the background noise is reduced by about 20% to 35%.

### Example 3

Patients scheduled for clinically-indicated PET-CT scan are administered rifaximin 550 mg twice daily (BID) for seven days prior to their scans. Each patient has their prior scheduled PET-CT used as control; that is, their prior PET-CT scan is evaluated as a pre-treatment baseline for intestinal FDG uptake (paired controls). The PET-CT scans of a randomly-selected cohort of patients also scheduled for clinically-indicated PET-CT during the same time period are used as independent controls. Cecal SUVMax (maximum-pixel-value standardized uptake value) and SUVAvg (average standardized uptake value) for all scans are measured by a radiologist blinded to treatment/control status and normalized to liver SUV, which is selected as the reference for normal tissue activity. A qualitative score of colonic uptake (0-4) is also assessed. Statistical analysis was performed by comparing mean SUV cecum/liver by paired t-test (pre & post) or unpaired t-test (treated & controls). The effect of rifaximin on a global measure of colonic SUV is also measured.

Patients who receive rifaximin for seven days prior to PET-CT scans exhibit a significant reduction in FDG uptake when compared to their pre-rifaximin scan. Accordingly, pre-treatment with rifaximin for seven days prior to PET-CT scans can reduce physiological F-18 uptake in the intestinal tract.

Embodiments are directed to reducing uptake of a substrate by metabolically active cells in a subject, comprising administering to the subject a composition comprising rifaximin. In some embodiments, the metabolically active cells are non-metastatic cells such as intestinal bacteria and colonic bacteria. In some embodiments, the metabolically active cells include, for example, E. coli. In some embodiments, the subject is administered 1100 mg of rifaximin per day. In some embodiments, the subject is administered 550 mg rifaximin twice per day. In some embodiments, the composition comprising rifaximin is administered to the subject about seven days prior to administration of the substrate. In some embodiments, the composition comprising rifaximin is administered to the subject at least about seven days prior to administration of the substrate. In some embodiments, the substrate is FDG.

Embodiments are also directed to reducing the background false positive noise of a PET scan of a subject, comprising administering to the subject a composition comprising rifaximin. In some embodiments, the subject is administered 1100 mg of rifaximin per day. In some embodiments, the subject is administered 550 mg rifaximin twice per day. In some embodiments, the composition comprising rifaximin is administered to the subject about seven days prior to administration of the substrate. In some embodiments, the composition comprising rifaximin is administered to the subject at least about seven days prior to administration of the substrate. In some embodiments, the background noise is reduced by about 10% to 50%. In some embodiments, the background noise is reduced by about 15% to 40%. In some embodiments, the background noise is reduced by about 20% to 35%.

### Example 4

Patients scheduled for clinically-indicated PET-CT scan are administered rifaximin 550 mg twice daily (BID) for between 48 hours to five days prior to their scans. Each patient has their prior scheduled PET-CT used as control; that is, their prior PET-CT scan is evaluated as a pre-treatment baseline for intestinal FDG uptake (paired controls). The PET-CT scans of a randomly-selected cohort of patients also scheduled for clinically-indicated PET-CT during the same time period are used as independent controls. Cecal SUVMax (maximum-pixel-value standardized uptake value) and SUVAvg (average standardized uptake value) for all scans are measured by a radiologist blinded to treatment/control status and normalized to liver SUV, which is selected as the reference for normal tissue activity. A qualitative score of colonic uptake (0-4) is also assessed. Statistical analysis was performed by comparing mean SUV cecum/liver by paired t-test (pre & post) or unpaired t-test (treated & controls). The effect of rifaximin on a global measure of colonic SUV is also measured.

Patients who receive rifaximin for between 48 hours to 5 days prior to PET-CT scans exhibit a significant reduction in FDG uptake when compared to their pre-rifaximin scan. Accordingly, pre-treatment with rifaximin for between 48 hours to 5 days prior to PET-CT scans can reduce physiological F-18 uptake in the intestinal tract.

Embodiments are directed to reducing uptake of a substrate by metabolically active cells in a subject, comprising administering to the subject a composition comprising rifaximin. In some embodiments, the metabolically active cells are non-metastatic cells such as intestinal bacteria and colonic bacteria. In some embodiments, the metabolically active cells include, for example, E. coli. In some embodiments, the subject is administered 1100 mg of rifaximin per day. In some embodiments, the subject is administered 550 mg rifaximin twice per day. In some embodiments, the composition comprising rifaximin is administered to the subject between about 48 hours to five days prior to administration of the substrate. In some embodiments, the substrate is FDG.

Embodiments are also directed to reducing the background false positive noise of a PET scan of a subject, comprising administering to the subject a composition comprising rifaximin. In some embodiments, the subject is administered 1100 mg of rifaximin per day. In some embodiments, the subject is administered 550 mg rifaximin twice per day. In some embodiments, the composition comprising rifaximin is administered to the subject between about 48 hours to five days prior to administration of the substrate. In some embodiments, the background noise is reduced by about 10% to 50%. In some embodiments, the background noise is reduced by about 15% to 40%. In some embodiments, the background noise is reduced by about 20% to 35%.

### Example 5

Patients scheduled for clinically-indicated PET-CT scan are administered rifaximin 1100 mg per day for 48 hours to seven days prior to their scans. Each patient has their prior scheduled PET-CT used as control; that is, their prior PET-CT scan is evaluated as a pre-treatment baseline for intestinal FDG uptake (paired controls). The PET-CT scans of a randomly-selected cohort of patients also scheduled for clinically-indicated PET-CT during the same time period are used as independent controls. Cecal SUVMax (maximum-pixel-value standardized uptake value) and SUVAvg (average standardized uptake value) for all scans are measured by a radiologist blinded to treatment/control status and normalized to liver SUV, which is selected as the reference for normal tissue activity. A qualitative score of colonic uptake (0-4) is also assessed. Statistical analysis was performed by comparing mean SUV cecum/liver by paired t-test (pre & post) or unpaired t-test (treated & controls). The effect of rifaximin on a global measure of colonic SUV is also measured.

Patients who receive rifaximin for 48 hours to seven days prior to PET-CT scans exhibit a significant reduction in FDG uptake when compared to their pre-rifaximin scan. Accordingly, pre-treatment with rifaximin for 48 hours to seven days prior to PET-CT scans can reduce physiological F-18 uptake in the intestinal tract.

Embodiments are directed to reducing uptake of a substrate by metabolically active cells in a subject, comprising administering to the subject a composition comprising rifaximin. In some embodiments, the metabolically active cells are non-metastatic cells such as intestinal bacteria and colonic bacteria. In some embodiments, the metabolically active cells include, for example, E. coli. In some embodiments, the subject is administered 1100 mg of rifaximin per day. In some embodiments, the composition comprising rifaximin is administered to the subject between about 48 hours to seven days prior to administration of the substrate. In some embodiments, the substrate is FDG.

Embodiments are also directed to reducing the background false positive noise of a PET scan of a subject, comprising administering to the subject a composition comprising rifaximin. In some embodiments, the subject is administered 1100 mg of rifaximin per day. In some embodiments, the composition comprising rifaximin is administered to the subject between about 48 hours to seven days prior to administration of the substrate. In some embodiments, the background noise is reduced by about 10% to 50%. In some embodiments, the background noise is reduced by about 15% to 40%. In some embodiments, the background noise is reduced by about 20% to 35%.

### Example 6

Patients scheduled for clinically-indicated PET-CT scan are administered rifaximin as a solid dispersion, embodiments of which are described in WO 2012/009388, for 48 hours to seven days prior to their scans. The solid dispersion contains between about 10 mg to about 100 mg rifaximin, and it is administered from about once per day up to about three times daily (TID). Each patient has their prior scheduled PET-CT used as control; that is, their prior PET-CT scan is evaluated as a pre-treatment baseline for intestinal FDG uptake (paired controls). The PET-CT scans of a randomly-selected cohort of patients also scheduled for clinically-indicated PET-CT during the same time period are used as independent controls. Cecal SUVMax (maximum-pixel-value standardized uptake value) and SUVAvg (average standardized uptake value) for all scans are measured by a radiologist blinded to treatment/control status and normalized to liver SUV, which is selected as the reference for normal tissue activity. A qualitative score of colonic uptake (0-4) is also assessed. Statistical analysis was performed by comparing mean SUV cecum/liver by paired t-test (pre & post) or unpaired t-test (treated & controls). The effect of rifaximin on a global measure of colonic SUV is also measured.

Patients who receive rifaximin for 48 hours to seven days prior to PET-CT scans exhibit a significant reduction in FDG uptake when compared to their pre-rifaximin scan. Accordingly, pre-treatment with rifaximin for 48 hours to seven days prior to PET-CT scans can reduce physiological F-18 uptake in the intestinal tract.

Embodiments are directed to reducing uptake of a substrate by metabolically active cells in a subject, comprising administering to the subject a composition comprising rifaximin. In some embodiments, the metabolically active cells are non-metastatic cells such as intestinal bacteria and colonic bacteria. In some embodiments, the metabolically active cells include, for example, E. coli. In some embodiments, the composition is administered as a solid dispersion, wherein the solid dispersion contains from about 10 mg to about 100 mg rifaximin. In some embodiments, the composition comprising rifaximin is administered to the subject between about 48 hours to seven days prior to administration of the substrate. In some embodiments, the composition comprising rifaximin is administered from about once per day up to about three times daily (TID). In some embodiments, the substrate is FDG.

Embodiments are also directed to reducing the background false positive noise of a PET scan of a subject, comprising administering to the subject a composition comprising rifaximin. In some embodiments, the subject is administered 1100 mg of rifaximin per day. In some embodiments, the composition is administered as a solid dispersion, wherein the solid dispersion contains from about 10 mg to about 100 mg rifaximin. In some embodiments, the composition comprising rifaximin is administered to the subject between about 48 hours to seven days prior to administration of the substrate. In some embodiments, the composition comprising rifaximin is administered from about once per day up to about three times daily (TID). In some embodiments, the background noise is reduced by about 10% to 50%. In some embodiments, the background noise is reduced by about 15% to 40%. In some embodiments, the background noise is reduced by about 20% to 35%.

### INCORPORATION BY REFERENCE

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of reducing the uptake of a substrate by non-metastatic cells in the intestinal tract of a subject, comprising administering a composition comprising rifaximin to the subject prior to administration of the substrate.

2. The method of claim 1, wherein the substrate uptake is reduced by about 10% to 50% relative to a subject that is not administered rifaximin.

3. The method of claim 2, wherein the substrate uptake is reduced by about 20% to 35% relative to a subject that is not administered rifaximin.

4. The method of claim 1, wherein the subject will undergo a positron emission tomography (PET) scan.

5. A method of reducing the risk of false positive diagnosis in a subject undergoing a positron emission tomography (PET) scan, comprising administering to the subject a composition comprising rifaximin prior to administration of the PET scan.

6. The method of claim 5, wherein administration of the composition results in a reduction in the uptake of a substrate by non-metastatic cells in the intestinal tract of the subject.

7. The method of claim 6, wherein the substrate uptake is reduced by about 10% to 50% relative to a subject that is not administered rifaximin.

8. The method of claim 7, wherein the substrate uptake is reduced by about 20% to 30% relative to a subject that is not administered rifaximin.

9. The method of claim 1 or claim 5, wherein administration of the composition results in a reduction in the risk of a false positive diagnosis relative to that of a subject that is not administered rifaximin prior to a PET scan.

10. The method of claim 1, wherein the substrate is a radiolabeled sugar analog.

11. The method of claim 10, wherein the substrate is radiolabeled with ¹¹C, ¹³N, ¹⁵O, ¹⁸F, ⁸²Rb, or combinations thereof.

12. The method of claim 10, wherein the substrate is 2-deoxy-2-[¹⁸F]fluoro-D-glucose (FDG).

13. (canceled)

14. The method of claim 1, wherein the subject is administered rifaximin at a dose of from about 10 mg to about 6000 mg; from about 50 mg to about 2500 mg BID; from about 50 mg to about 2000 mg TID; 200 mg TID; 200 mg BID or 200 mg QD.

15. The method of claim 14, wherein the subject is administered rifaximin at a dose of about 550 mg, 600 mg or 1650 mg TID, QD or BID.

16. The method of claim 14, wherein the subject is administered rifaximin at a dose of about 550 mg BID.

17. (canceled)

18. The method of claim 14, wherein the subject is administered rifaximin at a dose of about 1100 mg per day.

19. The method of claim 14, wherein the subject is administered rifaximin as a solid dispersion, wherein the solid dispersion comprises from about 10 mg to about 100 mg rifaximin.

20. The method of claim 1, wherein the subject is administered the composition about 24 hours to 7 or more days prior to administration of the substrate.

21. The method of claim 20, wherein the subject is administered the composition about 48 hours to 5 days prior to administration of the substrate.

22. The method of claim 20, wherein the subject is administered the composition about 48 hours prior to administration of the substrate.

23. The method of claim 20, wherein the subject is administered the composition at least about 5 days prior to administration of the substrate.
